(19) United States
(12) Patent Application Publication
(10) Pub. No.: US 2013/0326934 A1
(43) Pub. Date: Dec. 12, 2013

(54) SOLUBLE ACIDS FROM NATURALLY OCCURRING AQUEOUS STREAMS

(71) Applicants: Marcus Oliver Wigand, San Ramon, CA (US); Robert Martin CARLSON, San Ramon, CA (US); Mark Dean LOONEY, San Ramon, CA (US)

(72) Inventors: Marcus Oliver Wigand, San Ramon, CA (US); Robert Martin CARLSON, San Ramon, CA (US); Mark Dean LOONEY, San Ramon, CA (US)

(73) Assignee: CHEVRON U.S.A. INC., SAN RAMON, CA (US)

(21) Appl. No.: 13/491,925

(22) Filed: Jun. 8, 2012

(51) Int. Cl. C07C 51/00; C10L 1/00

(52) U.S. Cl. 44/307; 562/400

(57) ABSTRACT

Disclosed herein is a process for preparing transportation fuel and optionally a middle distillate, a diesel fuel, a heating oil, a jet fuel, a kerosene, an aviation gasoline, a gasoline fuel, or a lubricant base oil. The process includes the steps of (a) producing a naturally occurring aqueous fluid containing greater than 1 wt. % soluble carboxylic acids; (b) isolating at least a portion of the organic acids from the naturally occurring aqueous fluid; and (c) upgrading the isolated carboxylic acids. The step of upgrading the isolated carboxylic acids can include at least one of hydrotreating, hydrocracking, isomerization, esterification and FCC cracking. The aqueous fluid can then be used in processes for recovery of a mobile kerogen based product. Also disclosed herein is a process for preparing a biofuel comprising the organic acids isolated from naturally occurring aqueous fluid.

### RELATED APPLICATIONS

This application is related to U.S. application Ser. No. 13/335,409 (attorney docket number 70205.0216USU1), entitled “In-Situ Kerogen Conversion and Recovery” filed Dec. 22, 2011; U.S. application Ser. No. 13/335,525 (attorney docket number 70205.0216USU2), entitled “In-Situ Kerogen Conversion and Product Isolation” filed Dec. 22, 2011; U.S. application Ser. No. 13/335,607 (attorney docket number 70205.0216USU3), entitled “In-Situ Kerogen Conversion and Upgrading” filed Dec. 22, 2011; and U.S. application Ser. No. 13/335,673 (attorney docket number 70205.0216USU4), entitled “In-Situ Kerogen Conversion and Recycling” filed Dec. 22, 2011. The contents of all of these related applications are incorporated herein by reference in their entirety.

### BACKGROUND

If proponents of Hubbert peak theory are correct, world oil production will soon peak, if it has not done so already. Regardless, world energy consumption continues to rise at a rate that outpaces new oil discoveries. As a result, alternative sources of energy must be developed, as well as new technologies for maximizing the production and efficient consumption of oil. See T. Mast, Over a Barrel: A Simple Guide to the Oil Shortage, Greenleaf Book Group, Austin, Tex., 2005.

A particularly attractive alternative source of hydrocarbon products is oil shale, the attractiveness stemming primarily from the fact that oil can be “extracted” from the shale and subsequently refined in a manner much like that of crude oil. Technologies involving the extraction, however, must be further developed before oil shale becomes a commercially-viable source of energy. See J. T. Bartis et al, Oil Shale Development in the United States: Prospects and Policy Issues, RAND Corporation, Arlington, Va., 2005.

The largest known deposits of oil shale are found in the Green River Formation, which covers portions of Colorado, Utah, and Wyoming. Estimates on the amount of recoverable oil from the Green River Formation deposits are as high as 1.1 trillion barrels of oil—almost four times the proven oil reserves of Saudi Arabia. At current U.S. consumption levels (approximately 20 million barrels per day), these shale deposits could sustain the U.S. for another 140 years (Bartis et al.) At the very least, such shale resources could moderate the price of oil and reduce U.S. dependence on foreign oil. Associated with the Green River Formation are alkaline water sources rich in carbonates and hydrocarbons.

Oil shale typically consists of an inorganic component (primarily carbonaceous material, i.e., a carbonate), an organic component (kerogen) that can only be mobilized by breaking the chemical bonds in the kerogen, and frequently a second organic component (bitumen). Thermal treatment can be employed to break (i.e., “crack”) the kerogen into hydrocarbon chains or fragments, which are gas or liquids under retort conditions, and facilitate separation from the inorganic material. This thermal treatment of the kerogen is also known as “thermal upgrading” or “retorting,” and can be done at either the surface or in situ, where in the latter case, the fluids so formed are subsequently transported to the surface.

In some applications of surface retorting, the oil shale is first mined or excavated, and once at the surface, the oil shale is crushed and then heated (retorted) to complete the process of transforming the oil shale to a crude oil—sometimes referred to as “shale oil.” See, e.g., Shuman et al., U.S. Pat. No. 3,489,672. The crude oil is then shipped off to a refinery where it typically requires additional processing steps (beyond that of traditional crude oil) prior to making finished products such as gasoline, lubricant, etc. Note that various chemical upgrading treatments can also be performed on the shale prior to the retorting, See, e.g., So et al., U.S. Pat. No. 5,091,076.

A method for in situ retorting of carbonaceous deposits such as oil shale has been described in Kvapil et al., U.S. Pat. No. 4,162,808. In this method, shale is retorted in a series of rubblized in situ retorts using combustion (in air) of carbonaceous material as a source of heat.

The Shell Oil Company has been developing new methods that use electrical heating for the in situ upgrading of subsurface hydrocarbons, primarily in subsurface formations located approximately 200 miles (320 km) west of Denver, Colo. See, e.g., Vinegar et al., U.S. Pat. No. 7,121,342; and Berchenko et al., U.S. Pat. No. 6,991,032. In such methods, a heating element is lowered into a well and allowed to heat the kerogen over a period of approximately four years, slowly converting (upgrading) it into oils and gases, which are then pumped to the surface. To obtain even heating, 15 to 25 heating holes could be drilled per acre. Additionally, a ground-freezing technology to establish an underground barrier around the perimeter of the extraction zone is also envisioned to prevent groundwater from entering and the retorting products from leaving. While the establishment of “freeze walls” is an accepted practice in civil engineering, its application to oil shale recovery still has unknown environmental impacts. Additionally, the Shell approach is recognized as an energy intensive process and requires a long timeframe to establish production from the oil shale.

In view of the aforementioned limitations of the above methods, simpler and more cost-effective methods of extracting products from the oil shale consisting of kerogen in an inorganic matrix would be extremely useful.

As described above, associated with the deposits of oil shale are large water sources. These water sources are alkaline and contain solubilized minerals, including sodium carbonates. The water sources also contain solubilized organics, including organic carboxylic acids. These waters are often referred to as black water or Trona water. In particular, the Green River Formation deposits of oil shale are associated with what is referred to as Trona water or black Trona water. The water associated with the Green River oil sale occurs in unusual porous and permeable layers of variable richness in the Wilkins Peak member of the Green River Formation. The black color of the water associated with oil shale is caused by carboxylic acids dissolved in alkaline sodium carbonate solutions. This water was discovered during drilling operations in the northern part of the Green River Basin. These waters could be a valuable potential source for hydrocarbons and minerals. Methods for recovery of these valuable products from the water sources associated with the deposits of oil shale are needed.

### SUMMARY OF THE INVENTION

The present invention relates to recovery and production of organic acids occurring in aqueous fluids, most of which are subterranean. These organic acids can be recovered and upgraded to produce valuable products, including fuels and lubricants. The recovered organic acids can also be blended with biofuel.

In one embodiment is provided a process for preparing a transportation fuel. The process comprises (a) producing a naturally occurring aqueous fluid containing greater than 1 wt. % soluble carboxylic acids; (b) isolating at least a portion of the organic acids from the naturally occurring aqueous fluid; and (c) upgrading the isolated carboxylic acids. The upgrading step of the process can include at least one of hydrotreating, hydrocracking, isomerization, esterification and FCC cracking. The process can also further include a step of preparing at least one of a middle distillate, a diesel fuel, a heating oil, a jet fuel, a kerosene, an aviation gasoline, a gasoline fuel, or a lubricant base oil.

The isolated organic acids can also be blended with a biofuel before upgrading. The blended product can be upgraded and provided as a biofuel.

### DETAILED DESCRIPTION OF THE INVENTION

In accordance with this detailed description, the following abbreviations and definitions apply. It must be noted that as used herein, the singular forms “a”, “an”, and “the” include plural referents unless the context clearly dictates otherwise. Thus, for example, reference to an “organic acid” includes a plurality of such.

As used herein, the terms “hydrocarbon” or “hydrocarbonaceous” or “petroleum” are used interchangeably to refer to organic material originating from oil shale, coal, tar sands, crude oil, natural gas, naturally occurring aqueous fluids or biological processes. Carbon and hydrogen are major components of hydrocarbons; minor components, such as oxygen, sulfur and nitrogen may also occur in the hydrocarbons. The hydrocarbon fraction may include both aliphatic and aromatic components. The aliphatic component can further be divided into acyclic alkanes, referred to as paraffins, and cycloalkanes, referred to as naphthenes. A paraffin refers to a cyclic, linear or branched saturated hydrocarbon. For example, a C₈ paraffin is a cyclic, linear or branched hydrocarbon having 8 carbon atoms per molecule. Normal octane, methylheptane, dimethylhexane, and trimethylpentane are examples of C₈ paraffins. A paraffin-rich feed comprises at least 10 wt %, at least 20 wt % or even at least 30 wt % paraffins. For example, a C₈ rich paraffinic feedstock contains at least 10 wt. % C₈ hydrocarbons.

As disclosed herein, boiling point temperatures are based on the ASTM D-2887 standard test method for boiling range distribution of petroleum fractions by gas chromatography, unless otherwise indicated. The mid-boiling point is defined as the 50% by volume boiling temperature, based on an ASTM D-2887 simulated distillation.

As disclosed herein, carbon number values (i.e., C₅, C₆, C₈, C₉ and the like) generally refers to a number of carbon atoms within a molecule. Carbon number ranges as disclosed herein (e.g., C₈ to C₁₂) refer to molecules having a carbon number within the indicated range (e.g., between 8 carbon and 12 carbon atoms), including the end members of the range. Likewise, an open ended carbon number range (e.g., C₃₅+) refers to molecules having a carbon number within the indicated range (e.g., 35 or more carbon atoms), including the end member of the range. Unless otherwise specified, carbon number distributions are determined by true boiling point distribution and gas liquid chromatography.

Unless otherwise specified, feed rate to a catalytic reaction zone is reported as the volume of feed per volume of catalyst per hour. In effect, the feed rate as disclosed herein, referred to as liquid hourly space velocity (LHSV), is reported in reciprocal hours (i.e., hr⁻¹).

As used herein, the value for octane refers to the research octane number (RON), as determined by ASTM D2699.

The term “surface facility” as used herein is any structure, device, means, service, resource or feature that occurs, exists, takes place or is supported on the surface of the earth. Organic acids that are generated in the process are isolated from the naturally occurring aqueous fluid in surface facilities.

“Organic acid” is a term used herein to denote a molecular entity containing at least one carboxylic acid functional group, either in the non-ionized form (e.g., —COOH), in the ionized form (e.g., —COO—), or salts thereof. The term “organic acid” is meant to encompass a high molecular weight kerogen fragment (e.g., a molecular mass of up to 12,000 to 15,000 daltons and higher) comprising at least one carboxylic acid functional group. The term “organic acid” is further meant to encompass a low molecular weight acid, including a monoacid such as acetic acid and a diacid such as oxalic acid. As used herein, the term “monoacid” is used to denote having one carboxylic acid functional group per molecule. Likewise, the term “diacid” denotes two, and “triacid” denotes three carboxylic acid functional groups per molecule.

Hydrocracking is a chemical reaction of liquid feed materials, such as hydrocarbons, petroleum or biologically derived material, in the presence of hydrogen and one or more catalysts, resulting in product molecules having reduced molecular weight relative to that of the liquid feed materials. Additional reactions, including olefin and aromatic saturation and heteroatom (including oxygen, nitrogen, sulfur and halogen) removal may also occur during hydrocracking.

The term “in situ,” as used herein refers to the environment of the subsurface shale formation.

The term “commercial petroleum-based products,” as used herein, refers to commercial products that include, but are not limited to, gasoline, aviation fuel, diesel, lubricants, petrochemicals, and the like. Such products can also include common chemical intermediates and/or blending feedstocks.

Biofuels are produced from sustainable or renewable feedstock. Biofuel is derived from renewable plant and animal materials. Examples of biofuels include ethanol (often made from corn and sugarcane), biodiesel (vegetable oils and liquid animal fats), green diesel (derived from algae and other plant sources) and biogas (methane derived from animal manure and other digested organic material). By regulation it is possible to blend 40% of syncrude derived from Green River oil shale with biofuel and still identify it as a biofuel.

“Optional” or “optionally” means that the subsequently described event or circumstance may, but need not, occur, and that the description includes instances where the event or circumstance occurs and instances in which it does not.

In one embodiment, the process of the invention comprises recovering organic acids from naturally occurring aqueous fluids. In one embodiment, the process also comprising converting the organic acids via an upgrading process into fuel and lubricant products. The aqueous fluids can also be used in processes for mobilizing kerogen from subsurface shale formations.

In another embodiment, the process comprises blending the organic acids recovered from naturally occurring aqueous fluids with biofuel; upgrading the blend; and providing a biofuel product.

### Producing

In one embodiment, organic acids occur in naturally occurring aqueous fluids. Most of these naturally occurring aqueous fluids are subterranean; they can be recovered by penetrating the aquifer with a well and pumping the aqueous fluid to the surface for isolation and upgrading of the organic acids contained therein. While the source of the organic acids in the naturally occurring aqueous fluids has not been specifically identified, it is believed that at least a portion of the organic acids are derived from the conversion of subterranean kerogen over geological timeframes. These naturally occurring aqueous fluids that contain significant organic acids contents are known by a number of different names. The terms “Trona water” and “black water” are frequently applied to at least one source. The se naturally occurring aqueous fluids can also be sources for minerals.

By “naturally occurring” is meant originating from and/or isolated from a source in nature, e.g., from a subterranean aquifer or from a surface body of water such as a river or stream or from a pond or lake. It will be appreciated that a naturally occurring aqueous fluid or naturally occurring organic acids may be modified by, for example, the addition of organic or inorganic compounds, while the naturally occurring aqueous fluid or soluble organic acids are being produced, recovered or isolated. The aqueous fluid is water based, with substantial quantities of organic acids, which are stabilized in the fluid by high fluid pH. In general, the aqueous fluid is recovered from a natural source as a liquid phase solution, or as a slurry, suspension or emulsion.

The process of the invention includes a step of producing a naturally occurring aqueous fluid. In one embodiment, the producing step includes recovering an aqueous fluid from a body of water, such as stream, a river, a lake, or a pond. In one such embodiment, the aqueous fluid originates from a subterranean reservoir. In a further embodiment, the producing step includes recovering the aqueous fluid directly from a subterranean reservoir through a well penetrating the earth to the subterranean reservoir. Methods for forming the well, preparing the well for recovery of liquids and recovering the aqueous fluid from a subterranean reservoir are well known. In one embodiment, the produced aqueous fluid, either from a surface body of water or from a subterranean reservoir, is treated as described herein to isolate the organic acids contained therein.

These organic acids may be present in the naturally-occurring aqueous fluid at a concentration of greater than 1 wt %. In one embodiment, the organic acids are present at a concentration of greater than 2 wt. %; in another embodiment, greater than 5 wt. %. In another embodiment, the organic acids are present at a concentration in the range of from 1 wt. % to 50 wt. %; in another, from 1 wt. % to 40 wt. %; in another, from 1 wt. % to 30 wt. %. The ratio of organic acids to other hydrocarbons in the aqueous fluid will depend on the source of the hydrocarbons, but is expected to range from 10% organic acids to approaching 100 wt. % organic acids, with at most measurably trace amounts of other functional types.

In one embodiment, the naturally occurring aqueous fluid contains one or a mixture of inorganic components. In one such embodiment, at least one of the inorganic components contributes to increasing the solubility of the hydrocarbon component in the naturally occurring aqueous fluid. An exemplary naturally-occurring aqueous fluid has enhanced solubility for organic acids due to dissolved inorganic compounds that are alkaline in aqueous solution. In one embodiment, the naturally occurring aqueous fluid has a pH of at least 7.5, or at least 8.0, or at least 8.5, or at least 10. In one embodiment, the naturally occurring aqueous fluid has a pH in the range of between 8.5 and 14. As such, the naturally occurring aqueous fluid is alkaline. Exemplary alkaline inorganic materials solubilized in the aqueous fluids include, for example, at least one alkaline material selected from a carbonate, bicarbonate, and oxide, and a hydroxide of, for example, sodium, potassium, calcium, and magnesium. In one embodiment, the aqueous fluid comprises an alkaline material selected from the group consisting of sodium carbonate, sodium bicarbonate, and sodium hydroxide. At least a portion of the alkaline material may be in the form of carbonate and/or bicarbonate minerals. These carbonates can also be valuable products for recovery. An illustrative aqueous fluid contains a molar ratio of carbonate to bicarbonate in the range from 5:95 to 95:5; or in the range from 10:90 to 90:10; or in the range from 25:75 to 75:25.

### Organic Acids

In one embodiment, the naturally occurring aqueous fluid contains C₃₅− organic acids and C₃₅+ organic acids. The organic acids may be saturated, unsaturated, or polyunsaturated. In one embodiment, at least a portion of the organic acids are branched; the branching functional groups may be paraffinic, olefinic or cyclic. Cyclic branching functional groups may be saturated, unsaturated or aromatic. The organic acids may also contain nitrogen and/or sulfur atoms. In one embodiment, the organic acids are monoacids (a single carboxyl functional group in non-ionized or ionized form per molecular unit), or diacids (two carboxyl functional groups per molecular unit), or triacids (three carboxyl functional groups per molecular unit), or higher. Large molecular fragments, including fragments having a molecular mass of up to 12,000 to 15,000 Daltons or higher, may have multiple carboxyl functional groups that serve to render these fragments mobile in an aqueous medium. These high molecular weight fragments are generally mobilized in the fluid as a slurry, rather than as a pure solution. Under some conditions, the organic acids are present as salts. An exemplary sodium salt of an organic acid contains the carboxyl function group represented by —COO—Na+.

The molecular weight of the organic acids covers a very wide range, including from low molecular weight acids, such as the monoacid, acetic acid, and the diacid, oxalic acid, to high molecular weight fragments, having a molecular mass of up to 15,000 daltons or higher, and comprising at least one carboxylic acid functional group. Such high molecular weight acids are soluble in, or otherwise mobile in, high pH solutions, such as solutions having a pH of at least 7; or at least 8; or at least 8; or in the range of between 12 and 14. Accordingly, in one embodiment, at least 10 wt. %, or at least 30 wt. %, or at least 50 wt. % of the organic acids in the mobile kerogen-based product is in the C₃₅+ range. In one embodiment, at least 20 wt. % of the C₃₅+ organic acids has a molecular mass number of greater than 1000 daltons.

In one embodiment, a significant fraction of the organic acids are also in the C₆ to C₂₀ carbon number range. In one such embodiment, at least 10 wt. % of the organic acids are in the C₆ to C₂₀ range, or in the C₆ to C₁₆ range, or in the C₈ to C₁₄ range or in the C₈ to C₁₂ range. In one such embodiment, at least 20 wt. % of the C₃₅— organic acids is in the C₈ to C₁₂ range.

In general, the naturally occurring aqueous fluid contains at least one of the following organic acids and hydrocarbons: monoacids, diacids, branched monoacids, branched diacids, isoprenoid acids, hopanoic acids, gamma keto acids, keto monoacids, keto diacids, and n-alkanes. In the range from 10 wt. % to 90 wt. % of the C₃₅— organic acids in the aqueous fluid are monoacids. In one such embodiment, in the range from 10 wt. % to 50 wt. % of the C₃₅— organic acids are monoacids. Likewise, in the range from 10 wt. % to 90 wt. % of the C₃₅— organic acids are diacids. In one embodiment, at least 30 wt. % of the C₃₅— organic acids are diacids. In one such embodiment, in the range from 30 wt. to 90 wt. % of the C₃₅— organic acids are diacids. In one embodiment, in the range from 1 wt. % to 30 wt. %, or in the range from 1 wt. % to 20 wt. %, or in the range from 1 wt. % to 10 wt. % of the C₃₅— organic acids are gamma keto acids.

### Isolating Organic Acids

The organic acids are isolated from the naturally occurring aqueous fluid in surface facilities. In one embodiment, isolating the organic acids involves one or more of pH adjustment or titration, esterification, extraction, distillation, membrane separation, froth flotation, phase separation, electrostatic separation, coalescence, biological processes, thermal separation processes, steam distillation, in any order.

### pH Adjustment or Titration

Reducing the pH of the naturally occurring aqueous fluid generally involves adding an acid to the fluid. Suitable acids for this step include the mineral acids, such as sulfuric acid, hydrochloric acid, nitric acid, phosphoric acid or mixtures thereof. In some embodiments, addition of CO₂ to the naturally occurring aqueous fluid lowers the pH sufficiently to produce a second phase of the high molecular weight organic acids, which may be liquid or solid. In one embodiment, sufficient acid is added to the naturally occurring aqueous fluid to reduce the pH of the product to less than 7.0, or within a range of 1.5 to 6.5, or within a range of 2.0 to 6.0. In one embodiment, the pH is lowered in a single step to produce an organic acid syncrude. In other embodiments, the pH is lowered in multiple steps, with organic acid products being isolated and recovered after each step.

The amount of acid that is added is controlled by a number of factors that are specific to the particular process, including the target pH of the acidified product, the chemical character of the dissolved organic acids and the composition of the product prior to an acidification step. For example, a naturally occurring aqueous fluid that contains a carbonate/bicarbonate mixture in a buffering effective amount will generally require a greater amount of acid for acidifying the fluid. Acidifying conditions may include, for example, a temperature in the range from 0° C. to 200° C.; or in the range from 10° C. to 150° C.; or in the range from 20° C. to 100° C.; or even in the range from 25° C. to 75° C.

During pH titration, the non-ionized organic acids which are formed are less soluble in the aqueous naturally occurring aqueous fluid than are the ionized organic acids; these relatively insoluble organic acids form a separate liquid (or solid) phase from the aqueous fluid, and may be isolated from the fluid using conventional separation methods.

High molecular weight organic acids, including kerogen fragments that form either an emulsion or a solution in highly alkaline aqueous fluids, may be separated from the aqueous fluids at pH values of at least 7.5. Under these conditions, gaseous CO₂ that is provided to the naturally occurring aqueous fluid is adequate for reducing the pH such that these high molecular weight organic acids separate from the naturally occurring aqueous fluid as a separate liquid, or solid, phase. In one embodiment, at least a portion of the gaseous CO₂ used for pH titration is recovered from a decarboxylation process or from the pH titration process.

### Stepwise pH Adjustment or Titration

In one embodiment, the process of isolating organic acids is modified to facilitate the separation of product organic acids. Fractional neutralization is an example of such a process. During fractional neutralization, the naturally occurring aqueous fluid is treated in sequential steps; the treatment process converts increasingly larger amounts of relatively more soluble ionized organic acids to relatively more insoluble non-ionized organic acids. The organic acids which are isolated during each stage of the fractional neutralization process are characterized by a particular set of chemical and physical properties, such as molecular weight, number of carboxylate groups per molecule, and the olefinicity of the organic acids. In an illustrative example of a fractional neutralization process, a naturally occurring aqueous fluid is treated with an acid to reduce the pH of the naturally occurring aqueous fluid to a target value. Dissolved reaction products that are relatively insoluble in aqueous fluid at that pH form a separate phase, at least a portion of which is isolated from the remaining naturally occurring aqueous fluid. The organic acids can be fractionated by pH based on their chain length.

Two phase liquid-liquid separation methods are known. In one embodiment, the separation is facilitated by adding an organic extractant, either before, during or after acidification, for extracting the relatively insoluble products from the aqueous naturally occurring aqueous fluid. After recovery of the separate organic acid liquid phase, the naturally occurring aqueous fluid is further acidified to progressively lower pH values, and the separate organic acid liquid phase that is produced with each step of acidification is recovered, optionally with the use of additional amounts of one or more organic extractants.

When carbonate or bicarbonate containing materials are present in the aqueous fluid, increasing the amount of added acid will also generally increase the amount of evolved carbon dioxide. In one embodiment, the generated carbon dioxide is captured and used, for example, for manufacturing and commercial applications, for enhancing the recovery of hydrocarbons from subsurface reservoirs, or disposed, for example in subsurface cavities, to reduce carbon dioxide emissions into the atmosphere.

### Extraction

In one embodiment, separation of the organic acids from an aqueous naturally occurring aqueous fluid is facilitated by contacting the product with an organic extractant, which is selected to be relatively insoluble in the aqueous fluid, to have a high solubility for organic acids, to have adequate chemical stability at the conditions of the extraction and to be relatively easily separated from the organic acids in a subsequent separation step. Contacting the product with the organic extractant produces a two phase liquid system, in which at least a portion of the acids in the aqueous product preferentially partition into the organic extractant. The organic extractant may be selected to preferentially partition a certain group of organic acids, so as to separate the organic acid product into groups by one or more desired physical and/or chemical property. The organic acids are separated from the organic extractant using known separation methods, including distillation.

Typical organic extractants include C₄ to C₂₁ hydrocarbons, including naphtha, diesel fuel, and gas oils; alcohols, including methanol, ethanol, propanol, butanol; aromatics, including benzene, toluene, the xylenes and alkyl substituted variations thereof; ethers; ketones; esters; amines; tetralin; n-methyl-2-pyrrolidone; tetrahydrofuran; 2-methyl tetrahydrofurane.

Extraction conditions are generally mild, including a temperature, for example in the range from 0° C. to 200° C.; or in the range from 10° C. to 150° C.; or in the range from 20° C. to 100° C.; or even in the range from 25° C. to 75° C.

### Froth Flotation

In one embodiment the organic acids can be isolated by froth flotation. Froth flotation techniques are well known to those of ordinary skill in the art. In froth flotation gaseous bubbles are introduced to aqueous fluid. The bubbles pass through the aqueous fluid agitating it. The organic acids are attracted to the bubbles and become concentrated in a foam at the surface of the aqueous fluid. The foam can be separated from the aqueous fluid and a pH reduction to recover the organic acids from the foam can be performed on a smaller scale. In addition, when using froth floatation to recover the organic acids, the aqueous fluid remains alkaline.

After removal of the organic acids, the alkaline aqueous fluid can be recycled to the formation and used in recovery a mobile kerogen based product as described in U.S. application Ser. No. 13/335,409 entitled “In-Situ Kerogen Conversion and Recovery”, U.S. application Ser. No. 13/335,525 entitled “In-Situ Kerogen Conversion and Product Isolation, U.S. application Ser. No. 13/335,607 entitled “In-Situ Kerogen Conversion and Upgrading, and U.S. application Ser. No. 13/335,673 entitled “In-Situ Kerogen Conversion and Recycling”. If using the alkaline aqueous fluid in these processes and integrated process with efficiencies is provided. Also, two product sources are accessed because organic acids are recovered from the aqueous fluid and a mobile kerogen based product is recovered from the subsurface shale.

As such, the froth flotation technique is advantageous because the pH reduction is performed on a smaller scale and after recover of the organic acids, the aqueous fluid remains alkaline and can be used in obtaining a mobile kerogen based product.

### Esterification

Organic acids may be isolated from the naturally occurring aqueous fluid using esterification. In general, the esters that are formed are less soluble in an aqueous fluid than are the organic acids. Insoluble esters are separated from the aqueous fluid by liquid-liquid separation techniques.

### Recovery of Minerals

The aqueous fluid is also a source of minerals. The aqueous fluid is rich in carbonates/bicarbonates. These minerals can be removed and recovered as salts. If the aqueous fluid has not been acidified, the minerals can be recovered by techniques well known to those of ordinary skill in the art. For example, the water can be evaporated to recover the minerals. In one embodiment, the organic acids can be recovered by froth flotation and the aqueous fluid can then be evaporated to collect the minerals. The minerals can be a valuable commercial product.

### Products

### Syncrude

It may be desirable to recover the organic acids from the naturally occurring aqueous fluid as a syncrude which contains at least a portion of the organic acids that are insoluble in aqueous fluid at a pH of less than 2.0 or at a pH of less than 1.5. The syncrude prepared in this way will include organic acids of C₁₂ and higher, with deceasing amounts of organic acids of carbon number below C₁₂, depending on the particular acids. In some embodiments, the syncrude will include organic acids in the range of C₁₂ to C₃₅, or C₁₂ to C₃₀, or C₁₂ to C₂₀. Syncrude is a suitable feedstock for refining, petrochemical and power generating facilities. C₁₀ to C₃₅ organic acids

The C₁₀ to C₃₅ organic acids are important for preparing fuels and lubricants, petrochemicals and petrochemical feedstocks, refinery raw materials and process oils. Many specialized products have been proposed for organic acids in this molecular weight range. For example, refinery processes such as hydroprocessing, hydrogenation, saturation, hydrotreating, hydrocracking, isomerization, fluid catalytic cracking, thermal cracking, esterification, oligomerization, reforming, alkylation, denitrification and desulfurization are suitable for upgrading the organic acids to commercially valuable products.

### Upgrading the Organic Acids

As described, organic acids and other kerogen reaction products are separated from the naturally occurring aqueous fluid. In further embodiments, the extracted kerogen-based product is upgraded to yield one or more commercial petroleum-based products. Various techniques common in the industry (e.g., hydroprocessing, hydrogenation, saturation, hydrotreating, hydrocracking, isomerization, fluid catalytic cracking, thermal cracking, esterification, oligomerization, reforming, alkylation, denitrification and desulfurization) may be employed to obtain a desired commercial product from the recovered organic acids. Such upgrading is largely dependent on the nature of the recovered product relative to the commercial product that is desired.

The organic acids are used, for example, in the production of fuels, lubricant and lubricant base oils, polymers, pharmaceuticals, solvents, petrochemicals and food additives. In one embodiment, the acids are separated by e.g., chemical type or boiling range for specific chemical and petrochemical applications, including feedstock and end use applications. In one embodiment, at least some of the acids are used as feedstocks to make lubricating oil base stocks having a viscosity greater than or equal to 3 cSt at 40° C.; a pour point at or below 20° C., or at or below 0° C.; and a VI at least 70, or at least 90, or at least 120. It is optionally used with additives, and/or other base oils, to make a finished lubricant. The finished lubricants can be used in passenger car motor oils, industrial oils, and other applications. When used for passenger car motor oils, base oils meet the definitions of the current version of API Base Oil Interchange Guidelines 1509.

In one embodiment, at least some of the acids are used as feedstocks to make distillate fuels, generally boiling in the range of about C₅-700° F. (121°-371° C.) as determine by the appropriate ASTM test procedure. The term “distillate fuel” is intended to include gasoline, diesel, jet fuel and kerosene boiling range fractions. The kerosene or jet fuel boiling point range is intended to refer to a temperature range of about 280°-525° F. (138°-274° C.) and the term “diesel boiling range” is intended to refer to hydrocarbon boiling points of about 250°-700° F. (121°-371° C.). Gasoline or naphtha is normally the C₅ to 400° F. (204° C.) endpoint fraction of available hydrocarbons. The boiling point ranges of the various product fractions recovered in any particular refinery or synthesis process will vary with such factors as the characteristics of the source, local markets, product prices, etc. Reference is made to ASTM standards D-975, D-3699-83 and D-3735 for further details on kerosene, diesel and naphtha fuel properties.

### Hydrotreating

In one embodiment, the organic acids are upgraded in a hydrotreating reaction zone to remove heteroatoms such as oxygen, nitrogen and sulfur and to saturate olefins and aromatics. Hydrotreating conditions include a reaction temperature between 400° F.-900° F. (204° C.-482° C.), or between 650° F.-850° F. (343° C.-454° C.); a pressure between 500 to 5000 psig (pounds per square inch gauge) (3.5-34.6 MPa), or between 1000 to 3000 psig (7.0-20.8 MPa); a feed rate (LHSV) of 0.5 hr⁻¹ to 20 hr⁻¹ (v/v); and overall hydrogen consumption 300 to 2000 scf per barrel of liquid hydrocarbon feed (53.4-356 m³ H2/m³ feed). The hydrotreating catalyst will generally be a composite of a Group VI metal or compound thereof, and a Group VIII metal or compound thereof supported on a porous refractory base such as alumina. Examples of hydrotreating catalysts are alumina supported cobalt-molybdenum, nickel sulfide, nickel-tungsten, cobalt-tungsten and nickel-molybdenum. Other examples of hydrotreating catalysts contain a platinum group metal such as platinum and/or palladium. Typically such hydrotreating catalysts are presulfided.

### Hydrocracking

In one embodiment, the organic acids or reaction products derived from the organic acids are hydrocracked. The hydrocracking reaction zone is maintained at conditions sufficient to effect a boiling range conversion of the organic acids or derivatives thereof to the hydrocracking reaction zone, so that the liquid hydrocrackate recovered from the hydrocracking reaction zone has a normal boiling point range below the boiling point range of the feed. The hydrocracking step reduces the size of the hydrocarbon molecules, hydrogenates olefin bonds, hydrogenates aromatics, and removes traces of heteroatoms resulting in an improvement in fuel or base oil product quality.

Typical hydrocracking conditions include a reaction temperature between 400° F. and 950° F. (204° C.-510° C.) or between 650° F. and 850° F. (343° C.-454° C.); a reaction pressure between 500 and 5000 psig (3.5-34.5 MPa) or between 1500 and 3500 psig (10.4-24.2 MPa); a feed rate (in terms of volumes of feed at ambient conditions per volume of catalyst per hour) between 0.1 and 15 hr-1 (v/v) or between 0.25 and 2.5 hr-1; and hydrogen consumption 500 to 2500 scf per barrel of liquid hydrocarbon feed (89.1-445 m³ H₂/m³ feed). Generally, more severe conditions within these ranges will be used with higher boiling feedstocks and depending on whether gasoline, middle distillate or lubricating oil is desired as the primary economic product. The hydrocrackate is then separated into various boiling range fractions. The separation is typically conducted by fractional distillation preceded by one or more vapor-liquid separators to remove hydrogen and/or other tail gases.

The hydrocracking catalyst generally comprises a cracking component, a hydrogenation component and a binder. Such catalysts are well known in the art. The cracking component may include an amorphous silica/alumina phase and/or a zeolite, such as a Y-type or USY zeolite. The binder is generally silica or alumina. The hydrogenation component will be a Group VI, Group VII, or Group VIII metal or oxides or sulfides thereof, preferably one or more of molybdenum, tungsten, cobalt, or nickel, or the sulfides or oxides thereof. If present in the catalyst, these hydrogenation components generally make up from about 5% to about 40 wt. % of the catalyst. Alternatively, platinum group metals, especially platinum and/or palladium, may be present as the hydrogenation component, either alone or in combination with the base metal hydrogenation components molybdenum, tungsten, cobalt, or nickel. If present, the platinum group metals will generally make up from about 0.1% to about 2 wt. % of the catalyst.

### Isomerization

In one embodiment, the organic acids or reaction products derived from the organic acids are hydroisomerized. Typical hydroisomerization conditions are well known in the literature and can vary widely. Isomerization processes are typically carried out at a temperature between 200° F. and 700° F. or between 300° F. to 650° F., with a feed rate between 0.1 and 10 hr-1 or between 0.25 and 5 hr-1. Hydrogen is employed such that the mole ratio of hydrogen to hydrocarbon is between 1:1 and 15:1. Catalysts useful for isomerization processes are generally bifunctional catalysts that include a dehydrogenation/hydrogenation component and an acidic component. The acidic component may include one or more of amorphous oxides such as alumina, silica or silica-alumina; a zeolitic material such as zeolite Y, ultrastable Y, SSZ-32, Beta zeolite, mordenite, ZSM-5 and the like, or a non-zeolitic molecular sieve such as SAPO-11, SAPO-31 and SAPO-41. The acidic component may further include a halogen component, such as fluorine. The hydrogenation component may be selected from the Group VIII noble metals such as platinum and/or palladium, from the Group VIII non-noble metals such as nickel and tungsten, and from the Group VI metals such as cobalt and molybdenum. If present, the platinum group metals will generally make up from about 0.1% to about 2 wt. % of the catalyst. If present in the catalyst, the non-noble metal hydrogenation components generally make up from about 5% to about 40 wt. % of the catalyst.

### FCC

In one embodiment, the organic acids or reaction products derived from the organic acids are cracked in a fluid catalytic cracking unit (FCC). In such fluidized catalytic cracking, high molecular weight hydrocarbon liquids and vapors are contacted with hot, finely divided solid catalyst particles in an elongated riser or transfer line reactor. The transfer line is usually in the form of a riser tube and the contacting time is on the order of a few seconds, say from 0.5 to 8 seconds, and generally not over about 4 seconds. During this short period, catalysts at temperatures in the range about 1100° F. to 1400° F. are contacted with a hydrocarbon feedstock which is heated to a temperature of about 300° F. to 800° F. The reaction is one of essentially instantaneous generation of large volumes of gaseous hydrocarbons. The hydrocarbons and catalyst mixture flows out of the riser tube into a reactor vessel wherein the resultant gaseous hydrocarbons are taken off for distillation into various product fractions defined by boiling ranges. The spent catalyst is then separated in the reactor vessel and stripped of hydrocarbons by passing the catalyst through a stripper section which includes steam flowing up through the down-flowing catalyst usually for a period of 1 to 3 minutes. Catalyst is then returned to a regenerator where residual hydrocarbons, called “coke”, on the spent catalyst are burned off by passing a stream of an oxygen-containing gas, such as air, or oxygen-enriched air, through the catalyst until substantially all the carbon is burned from the particles. The heat generated in this regeneration step is used as a heat source to heat the catalyst and thus provide elevated temperatures needed for reaction with the incoming hydrocarbon feed. Regenerated hot catalyst is then recycled to the riser cracking zone wherein the feed is cracked to form more gaseous products. In one embodiment, the fluid catalytic cracking reaction of the organic acids is conducted at a temperature of between 1200° F. and 1400° F. (600° C. to 800° C.). In another embodiment the fluid catalytic cracking reaction of the organic acids is conducted at a temperature of between 250° C. and 490° C.

A typical catalyst comprises 10%-60% w/w of a solid acid, 0%-50% w/w of alumina, 0%-40% w/w of silica, the remainder being kaolin. The solid acid may be a zeolite of the ZSM type, a zeolite of the faujasite type, a zeolite of the mordenite type, silico-aluminum phosphate (SAPO) or aluminum phosphate (ALPO).

Many refineries operate a single reactor for fluid catalytic cracking of gas oil or residue. In some situations, refineries may employ an FCCU possessing two reactors working in simultaneous operation. In such units the streams of spent catalyst from two reactors are mixed in the same rectification section having a single regenerator to burn off coke deposited on the catalyst. In addition such reactors may function in an independent manner with respectively different types of charge and differing reaction temperatures. The reaction severity applied to each reactor may be totally different, making it possible to adjust them to preestablished operational objectives. In this manner it is possible to carry out the processing of the organic acids under milder conditions, at temperatures between 250° C. and 490° C., for the production of diesel oil having a cetane number exceeding 40, while simultaneously carrying out the processing of conventional heavy gas oils or residues under more severe conditions, employing a single catalyst flow for both processes.

### Esterification

The method of the present invention for converting the organic acids to esters involves blending suitable amounts of an alcohol, such as alcohol, and an acid catalyst with the organic acids. The reaction mixture is then subjected to conditions suitable for forming the esters.

A suitable amount of alcohol for the method of the present invention is an amount that is about 100% to about 470% of the theoretical amount needed to convert all organic acids into esters. The theoretical amount of alcohol needed to convert all organic acids into esters is defined as an equal number of alcohol molecules as that of organic acid molecules. In one embodiment, an amount of alcohol about 120% to about 300% of the theoretical amount is used in the method of the present invention. In another embodiment, an amount of alcohol between 100% and 150% of the theoretical amount is used. In still another embodiment, an amount of alcohol about 270% of the theoretical amount is used.

Suitable acid catalysts for converting organic acids to esters are known in the art. Any of these catalysts can be used in the process. Examples of these catalysts include but are not limited to sulfuric acid, hydrogen chloride and p-toluenesulfonic acid. When sulfuric acid is used, a suitable amount is about 0.1% to about 7.5% by weight in the reaction mixture. In one embodiment, the amount of sulfuric acid used is about 0.6% to about 5.8% or about 1.2%. When other acid catalysts are used, a skilled artisan either knows or can easily determine the suitable amount that can be added into the reaction mixture.

The speed that organic acids are converted to esters in the reaction mixtures described above is a function of the reaction temperature—the higher the reaction temperature, the higher the speed. In one embodiment, the reaction temperature used for esterifying the acids is at least 20° C.; or 25° C.; or 30° C.; or 35° C.; or 40° C.; or 45° C. with stirring. Most preferably, the reaction temperature is kept at about at least 50° C.; or 55° C.; or 60° C.; or 65° C. In one embodiment, the reaction temperature is the boiling point of the alcohol at the reaction pressure. The reaction pressure for esterification is generally equal to or slightly above (e.g., 200 psig) atmospheric pressure.

The reaction time needed to convert a desired percentage of organic acids into esters under specific amounts of alcohol, acid catalyst and reaction temperature can readily be determined by a skilled artisan. For example, a small sample of the reaction mixture can be taken at different time points and the organic acid level and ester level can be determined. Generally speaking, the reaction time range goes from 0.5 hr to 2.0 hr; or to 3.0 hr; or to 5.0 hr; or to 10 hr.

### Fuel and Lubricant Products

A “middle distillate” is a hydrocarbon product having a boiling range between 250° F. to 1100° F. (121° C. to 593° C.). The term “middle distillate” includes the diesel fuel, heating oil, jet fuel, and kerosene boiling range fractions. It may also include a portion of naphtha or light oil. A “naphtha” is a lighter hydrocarbon product having a boiling range between 100° F. to 400° F. (38° C. to 204° C.). A “light oil” is a heavier hydrocarbon product having a boiling range that starts near 600° F. (316° C.) or higher. A “jet fuel” is a hydrocarbon product having a boiling range in the jet fuel boiling range. The term “jet fuel boiling range” refers to hydrocarbons having a boiling range between 280° F. and 572° F. (138° C. and 300° C.). The term “diesel fuel boiling range” refers to hydrocarbons having a boiling range between 250° F. and 1000° F. (121° C. and 538° C.). The term “light oil boiling range” refers to hydrocarbons having a boiling range between 600° F. and 1100° F. (316° C. and 593° C.). The “boiling range” is the 10 vol % boiling point to the final boiling point (99.5 vol %), inclusive of the end points, as measured by ASTM D 2887-06a and ASTM D 6352-04.

A gasoline fuel also boils or distills over a range of temperatures. In general, a gasoline fuel will distill over the range of from about, room temperature to 437° F. (225° C.). This temperature range is approximate, of course, and the exact range will depend on the conditions that exist in the location where the automobile is driven. The distillation profile of the gasoline can also be altered by changing the mixture in order to focus on certain aspects of gasoline performance, depending of the time of year and geographic location in which the gasoline will be used. In one embodiment, the gasoline fuel is an aviation gasoline fuel.

A lubricant base oil boils in a temperature range of greater than 650° F. The boiling range of a specific lubricant base oil depends on the usage to which the base oil is employed. An exemplary light base oil boils in a temperature range of 650° to 900° F. In a further embodiment, a light base oil has a boiling range of approximately 650° F. to 900° F. (343° C. to 482° C.), a pour point not greater than about −5° C., and a kinematic viscosity at 100° C. of about 4 to about 5 mm2/s. An exemplary heavy base oil boils in the temperature range of 800° F. to 1100° F. An exemplary extra heavy base oil boils in the temperature range 90° F. to 1300° F.

### Biofuels

Biofuels are produced from sustainable or renewable feedstock. As such, Biofuel is derived from renewable plant and animal materials. Examples of biofuels include ethanol (made from corn and sugarcane), biodiesel (vegetable oils and liquid animal fats), green diesel (derived from algae and other plant sources) and biogas (methane derived from animal manure and other digested organic material). The green diesel derived from algae comprises carboxylic acids typically with shorter chain lengths than the organic acids recovered as described herein because the algae are producing carboxylic acids.

The organic acids recovered from naturally occurring aqueous fluids disclosed herein are also similar in composition to the syncrude derived Green River oil shale because both comprise carboxylic acids. By regulation it is possible to blend 40% of syncrude derived from Green River oil shale with biofuel and still identify it as a biofuel.

In another embodiment, the process as disclosed herein comprises blending the organic acids recovered from naturally occurring aqueous fluids with biofuel; upgrading the blend; and providing a biofuel product. The blend can be upgraded by esterification to provide the biofuel product.

The blend can contain 1 to 40 weight % organic acids isolated from the naturally occurring aqueous fluid and 99 to 60 weight % biofuel derived from renewable plant and animal materials.

### Variations

A variation (i.e., alternate embodiment) on the above-described process is the application of some or part of such above-described methods to alternative sources, i.e., low-permeability hydrocarbon-bearing (e.g., oil and gas) formations, in situ coal, in situ heavy oil, in situ oil sands, and the like. General applicability of at least some of the above-described invention embodiments to any hydrocarbon-bearing formation exists. Surface processing applications may include upgrading of oil shale, coal, heavy oil, oil sands, and other conventional oils with asphaltenes, sulfur, nitrogen, etc.

What is claimed is:

1. A process for preparing a transportation fuel, comprising the steps of:
a) producing a naturally occurring aqueous fluid containing greater than 1 wt. % soluble carboxylic acids;
b) isolating at least a portion of the organic acids from the naturally occurring aqueous fluid; and
c) upgrading the isolated carboxylic acids.

2. The process according to claim 1, wherein the naturally occurring aqueous fluid has a pH of at least 7.5.

3. The process according to claim 1, wherein the naturally occurring aqueous fluid has a pH in the range from 8.5 to 14.

4. The process according to claim 1, wherein the naturally occurring aqueous fluid further comprises an alkaline material selected from the group consisting of a carbonate, a bicarbonate, an oxide and a hydroxide.

5. The process according to claim 4, wherein the alkaline material is selected from the group consisting of sodium carbonate, sodium bicarbonate, and sodium hydroxide.

6. The process according to claim 5, further comprising recovering carbonates from the naturally occurring aqueous fluid.

7. The process according to claim 1, wherein the naturally occurring aqueous fluid contains in the range from 1 wt. % to 50 wt. % organic acids.

8. The process according to claim 1, further comprising producing a naturally occurring aqueous fluid containing C₃₅⁻ organic acids and C₃₅⁺ organic acids.

9. The process according to claim 8, wherein at least 50 wt. % of the organic acids are in the C₃₅⁺ range.

10. The process according to claim 8, wherein at least 20 wt. % of the C₃₅⁻ organic acids are in the C₈ to C₁₂ range.

11. The process according to claim 8, wherein at least 30 wt. % of the C₃₅⁻ organic acids are diacids.

12. The process according to claim 1, further comprising isolating at least a portion of the organic acids by reducing the pH of the naturally occurring aqueous fluid to produce a syncrude comprising organic acids.

13. The process according to claim 12, further comprising reducing the pH of the naturally occurring aqueous fluid product to a pH in the range from 2.0 to 6.5.

14. The process according to claim 12, further comprising reducing the pH stepwise to recover the organic acids.

15. The process according to claim 12, further comprising isolating at least a portion of the C₁₀⁺ organic acids from the naturally occurring aqueous fluid to produce a syncrude.

16. The process according to claim 12 further comprising reducing the pH of the naturally occurring aqueous fluid in the presence of an organic extraction fluid to produce an hydrocarbon enriched extractant that contains at least a portion of the organic acids.

17. The process according to claim 16, further comprising separating at least a portion of the organic acids from the hydrocarbon enriched extractant to produce a syncrude.

18. The process according to claim 1, further comprising isolating at least a portion of the organic acids by froth flotation.

19. The process according to claim 18, after isolating at least a portion of the organic acids by froth flotation further comprising using at least a portion of the aqueous fluid to recover a mobile kerogen based product.

20. The process according to claim 18, after isolating at least a portion of the organic acids by froth flotation further comprising recovering carbonates from the naturally occurring aqueous fluid.

21. The process according to claim 1, wherein the step of upgrading the isolated carboxylic acids includes at least one of hydrotreating, hydrocracking, isomerization, esterification and FCC cracking.

22. The process of claim 21, further comprising preparing at least one of a middle distillate, a diesel fuel, a heating oil, a jet fuel, a kerosene, an aviation gasoline, a gasoline fuel, or a lubricant base oil.

23. A process for preparing a biofuel comprising the steps of:
a) producing a naturally occurring aqueous fluid containing greater than 1 wt. % soluble carboxylic acids;
b) isolating at least a portion of the organic acids from the naturally occurring aqueous fluid; c) blending the organic acids with biofuel to provide a blend;
d) upgrading the blend; and
e) providing a biofuel product.

24. The process according to claim 23 wherein the blend is upgraded by esterification.

25. The process according to claim 23, wherein the blend comprises 1 to 40 weight % organic acids isolated from the naturally occurring aqueous fluid.
